(19) United States
(12) Patent Application Publication
(10) Pub. No.: US 2018/0207404 A1
(43) Pub. Date: Jul. 26, 2018

(54) DEFLECTABLE CATHETER WITH A FLEXIBLY ATTACHED TIP SECTION

(71) Applicant: BIOSENSE WEBSTER, INC., Diamond Bar, CA (US)

(72) Inventors: Seth J. WORLEY, Lancaster, PA (US); Shiva SHARAREH, Laguna Niguel, CA (US)

(21) Appl. No.: 15/935,839

(22) Filed: Mar. 26, 2018

Related U.S. Application Data
(63) Continuation of application No. 11/323,908, filed on Dec. 29, 2005.

(51) Int. Cl. A61M 25/01; A61B 18/14; A61B 17/00

(52) U.S. Cl. CPC A61M 25/0152; A61M 25/0147; A61M 25/0141; A61B 2017/00867; C08L 2201/12; A61B 18/1492

(57) ABSTRACT

A catheter for mapping and/or ablating a region of the heart includes an intermediate section that is connected to a tip assembly at a preset angle by a flexible preshaped section that is more flexible than the intermediate section. The flexible section may absorb displacement force applied to the tip assembly, such as when the tip assembly encounters uneven tissue surface, without displacing the intermediate section. The flexible section prevents excessive force from being applied to the tip assembly, reducing the risk of any of the following: a) mechanical perforation, b) steam pop, c) burying the tip assembly in the myocardium resulting in high temperatures, low energy delivery, thrombus formation and char formation.

### CROSS-REFERENCE TO RELATED APPLICATIONS

This application is a continuation of and claims priority to and the benefit of U.S. patent application Ser. No. 11/323,908, filed Dec. 29, 2005, the entire contents of which is incorporated herein by reference.

### FIELD OF THE INVENTION

The invention is directed to a catheter having a tip assembly for mapping and/or ablating regions of or near a heart.

### BACKGROUND OF THE INVENTION

For successfully mapping and/or ablating of regions of or near a heart, the tip assembly should ideally make contact with the surface of the heart without undue pressure. Excess pressure can result in mechanical trauma and damage to the heart and/or result in inadequate cooling of the tip of the catheter via the blood stream resulting in steam pops, char, coagulation, embolization and inadequate delivery of current for successfully ablation of the tissue. Catheter-based ablation is usually conducted within the heart. The inside of the heart is a complex three-dimensional structure with both concave, convex and tubular structures as well as multiple irregularities within the convex or on the concave structure. Further, the transition from concave to convex or into a tubular structure also results in changes in the surface contour of the inside of the heart. Depending on the mechanism of the cardiac arrhythmia, ablation may be required within a concave structure with both smooth and irregular surface contours, on a convex structure with both smooth and irregular surface contours or at the intersection of two or more complex structures. Ablation may also be required within, around and on complex three-dimensional contours created by the confluence of a concave, convex and a tubular structure which themselves may have smooth or irregular contours. Currently, available catheter technology attempts to address ablation of these various areas of the heart with an ablation tip assembly, the shape and direction of which is determined by puller wires or preset shapes where the bending modulus between the mapping or ablation section and the intermediate section is constant. The ability of the ablation section to accommodate the irregular contours within the heart is limited. Attempts to approximate and contact these complex surface contours with the ablation section may result in either no contact or excess surface pressure at the tip allow the ablation section to achieve the off axis angle from the intermediate section required to make surface contact.

A catheter design with an ablation or mapping tip assembly attached to the intermediate section by a flexible section with a modulus of elasticity that allows the tip assembly to be deflected without displacing the intermediate section of the catheter is important to successful and safe ablation. Further, it is recognized by one of ordinary skill in the art that specific arrhythmias associated with defined surface contours may be optimally addressed with a catheter where the flexible section connecting the intermediate section and the mapping and ablation assembly is off set from the intermediate section at a predefined angle either in the plane or out of the plane of the intermediate section.

Specific examples are provided below:

Atrial flutter and atrial fibrillation are common sustained cardiac arrhythmias. Atrial flutter occurs when the atria are stimulated to depolarize at 200-350 beats per minute and is maintained by macroreentrant circuits generated by electrical impulses traveling in a circular fashion around and in the atria. Atrial flutter results in poor atrial pumping since some parts of the atria are releasing while other parts are contracting. Fortunately, atrial flutter in the right atrium can be effectively treated by ablation of the inferior vena cava-tricuspid annulus isthmus to create a line of conduction block to interrupt the macroreentrant circuit. The region at or near the inferior vena cava-tricuspid annulus isthmus (hereinafter referred to as “the cava-tricuspid region”) can be difficult to map or ablate. Not only does the tissue in that region have a convex curvature contrary to the generally cavernous shape of the right atrium, the tissue surface is uneven. Therefore, it is desirable for a catheter entering the right atrium from the inferior vena cava (an entry that is below or inferior of the cava-tricuspid region) to have a catheter body that can be deflected to approximate the convex curvature of the cava-tricuspid region and a preshaped flexible off-axis catheter tip in the direction of deflection that can maintain contact with the uneven tissue as the tip is dragged along for mapping or ablation procedures.

To successfully ablate other ventricular and atrial arrhythmias, a focal lesion or a line of conduction block should be created in the generally concave cavity of the right atrium/left atrium/right ventricle/left ventricle (RA/LA/RV/LV). The tissue surface of these structures is generally uneven. Therefore, it is generally desirable to have a catheter body that can be deflected to approximate the concave curvature of the region and a pre-shaped flexible off-axis in-plane catheter tip that is opposite to the direction of deflection that can maintain contact with the uneven tissue as the tip is dragged along for mapping or ablation procedures.

In patients with refractory atrial fibrillation, the atria are stimulated to depolarize irregularly at 250-400 cycles per minute. Not every atrial activation results in a QRS complex (ventricular depolarization) because the AV Node acts as a filter. However, there are instances where it is desirable to create conduction block at or near the AV Bundle. This region of the right atrium, the atrioventricular Bundle (of His) near the Atrioventricular (AV) Node, poses similar challenges for mapping and ablation as the cava-tricuspid region. The region is also convex unlike the generally cavernous contour of the right atrium. Moreover, the atrium wall in this region is canted slightly to the anterior. Therefore, it is desirable for a catheter entering from the inferior vena cava (an entry that is also below or inferior of AV Bundle) to have a catheter body that can be deflected to approximate the convex curvature of the region and a preshaped flexible catheter tip that extends off-plane from the catheter body to circumvent the canted angle of tissue surface.

As with most catheter-based mapping and/or ablation procedures, the catheter section immediately proximal the tip may not be in contact with or supported/stabilized by any structure in the heart. Without supportive contact between this proximal catheter section and the tissue, motion of the heart during systole, diastole and respiration is not transmitted to this catheter section except by contact between tissue and the catheter tip. As the heart moves during systole, diastole and respiration, the contact pressure at the tip of the catheter may vary from excessive to nonexistent. In a catheter that approaches the atrium in a “forward” direction, the disparity between the generally motionless (or out of synch) catheter and the heart can make it difficult to maintain stable contact between the catheter tip and the atrium wall in a beating moving heart. An unsupported and thus unsynchronized catheter used in the atrium may be inadvertently advanced into the tricuspid valve. Also, nonuniform contours in the atrium can make it difficult to contact recessed areas without excess pressure on the protruding areas increasing the risk of perforation. In addition, the catheter position is maintained only by contact between the tip and the nonuniform contours causing the catheter tip to frequently lose contact with the tissue during ablation or mapping as the heart moves independently during systole, diastole and with respiration.

Accordingly, a desire exists for a catheter capable of effectively mapping and ablating complex regions such as those with a convex contour, such as the cava-tricuspid region and regions at or near the AV Bundle (of His). It is desirable that the catheter body is adapted to approximate the convex contour for improved access to the tissue of interest from the inferior vena cava, and that the catheter tip be able to maintain contact with the tissue surface without undue force and maintain stability during ablation and mapping despite the motion of beating heart in a breathing patient. A catheter of such design improves precision of mapping and/or ablation and minimizes risks of damage to the tissue, including tissue perforation and inadvertent entry into the tricuspid valve.

### SUMMARY OF THE INVENTION

The present invention is directed to a catheter having a flexibly attached tip assembly either on or off axis with the body of the catheter depending on the specific application for mapping and/or ablating regions of or near a heart. A catheter for mapping and/or ablating a region of the heart comprises a catheter body with an intermediate section that is connected to a tip assembly by a highly flexible pre-shaped section. The entire intermediate section may be deflected, or the intermediate section may comprise a deflectable proximal portion and a distal portion that is straight or curved. The highly flexible section may preset the tip assembly at an off-axis and/or off-plane angles from the intermediate section.

In a first embodiment, the tip assembly is in the same plane and same direction as deflection of the intermediate section. The intermediate section when deflected approximates a generally convex or concave region of the heart with the flexibly attached ablation/mapping assembly enabling improved and safer contact of this tip assembly with irregular contours contained in or on the concave or convex structures respectively.

In a second embodiment, the intermediate section when deflected approximates a generally concave region of various heart cavities including the right atrium/right ventricle/left atrium/left ventricle (RA/RV/LA/LV) and the preset angle of the flexible section presets the tip assembly in the same plane and opposite to the direction of deflection of the intermediate section enabling improved and safer contact of the tip assembly to the walls of the cavities for ablation and/or mapping.

In a third embodiment, the intermediate section when deflected approximates the generally convex region of the cavo-tricuspid isthmus and the preset angle of the flexible section presets the tip assembly off axis in the same plane and same direction as deflection of the intermediate section enabling improved and safer contact of the tip assembly with the cavo-tricuspid isthmus for ablation and/or mapping.

In a fourth embodiment, the intermediate section when deflected approximates the generally convex region of the His area and the flexible section presets the tip assembly out of plane with the intermediate section enabling improved and safer contact of the tip assembly to the Bundle of His region.

With any of the foregoing embodiments, the intermediate section may have a distal portion with shape memory to maintain a straight configuration or a curved configuration to improve approximation to the generally convex regions of the cavo-tricuspid and HIS regions or the generally concave regions of the RA/LA/LA/LV.

A high bending modulus of the flexible section that connects the tip section for mapping and ablation to the intermediate section enables the flexible section to absorb displacement force applied to the tip assembly, without displacing the intermediate section improving tissue contact when the tip assembly encounters uneven tissue surface. The high bending modulus of the flexible section allows the tip section to be displaced while limiting the force that the tip assembly can apply to the tissue reducing the risk of any of the following: direct mechanical perforation, steam pop perforation, and burying of the tip assembly in the myocardium resulting in high temperatures, low energy delivery, thrombus and char formation.

The specific application of the catheter of the present invention determines how the flexible section connects the tip assembly to the intermediate section. Parameters of the flexible section that determine the relationship between the tip assembly and the intermediate section includes the following: a) the off plane and/or off axis angle of the flexible section to the intermediate section, b) the flexibility of the flexible section c) the lateral stability of the flexible section, and d) the length of the flexible section.

In addition, the configuration of the intermediate section to which the tip assembly is flexibly attached also impacts on the function of the tip assembly. As mentioned, the entire intermediate section may be deflectable, or only its proximal section from which a straight or curved distal section extends. In addition, how the tip assembly flexibly extends in relation to the straight or curved distal section of the intermediate section also determines the specific application. The tip assembly can be flexibly attached: a) on or off axis with the straight or curved distal section of the intermediate section, b) when off axis whether the angle is in or out of plane with any curved distal section, c) when off axis whether the off axis tip assembly is in the direction of or opposite to the direction of any curved distal section, d)the length of the tip assembly beyond the flexible section, e)the construction of the tip assembly beyond the flexible section (e.g., irrigated or irrigated with or without temperature sensors or electromagnetic sensors), f)the force required to deflect the tip assembly off axis when the tip assembly is on axis, g)the force required to deflect the tip assembly toward the axis when the tip assembly is off axis.

In one embodiment, the present invention is directed to a catheter configured for mapping and ablating a generally convex region of the heart, such as the complex intersection of the inferior vena cava, RA, and RV at the cavo tricuspid isthmus. In a detailed embodiment, the catheter has an intermediate section and a tip assembly adapted for mapping and/or ablation that is attached to the intermediate section by a pre-shaped flexible section that allows the tip assembly to be moved generally independently of the intermediate section. In a more detailed embodiment, the catheter comprises an elongated flexible tubular catheter body having proximal and distal ends. The intermediate section is mounted on the distal end of the tubular body and deflected with a curvature that approximates the generally convex contour of the cavo-tricuspid isthmus or Bundle of His region of the right atrium. The tip assembly is attached to the end of the intermediate section by the flexible section which is configured with preset angles to extend the tip assembly off-axis and/or off-plane from the intermediate section so that the tip assembly can make suitable contact with the tissue surface of the isthmus and His region.

When deflected, the intermediate section of the catheter is configured to conform to the generally convex region so that motion of the heart is transferred to the catheter thereby providing stability to the tip assembly. The preshaped flexible section improves the ability of the tip assembly to access, contact and remain in contact with surrounding tissues of variable contour without undue pressure. Moreover, the preshaped flexible section may be reinforced to provide the tip assembly with stability in a selected angle. Accordingly, the catheter of the present invention has improved safety features and improved ablation and mapping capabilities.

In another embodiment of the convex design, the tip assembly is configured as an ablation assembly that may be irrigated, comprising a plurality of irrigation ports in between which an ablation coil electrode is wound. A porous covering, preferably made of expanded polytetrafluoroethylene, covers the coil electrode and irrigation ports. Fluid passes through the irrigation ports to the porous covering, which then disperses the fluid around the ablation assembly. This irrigation generally enables the creation of deeper lesions.

In use, the distal end of the catheter is inserted into a patient's body and advanced atraumatically into the right atrium of a patient's heart by entry from the inferior vena cava. The intermediate section is deflected onto or near a generally convex such as the cava-tricuspid isthmus or the His region. The off-axis angle of the tip assembly readily allows the tip assembly to contact the isthmus, whereas the off-plane angle of the tip assembly readily allows the tip assembly to contact the His region notwithstanding the awkward angle imposed on the catheter by the relative superior and/or anterior locations of these regions of interest relative to the inferior vena cava.

As the user operates the catheter and maneuvers the tip assembly, the deflected intermediate section advantageously synchronizes the catheter and the tip assembly with the motion of the heart while the pre-shaped flexible section advantageously allows the tip assembly to flex from the preset angle(s) as needed in order to remain in contact with the tissue. In one embodiment, as the tip assembly encounters protrusions and recesses while being dragged along the tissue surface, the tip assembly is jarred from its preset off axis angle but the flexible section allows the tip assembly to conform and ride along on the uneven surface without displacing the intermediate section.

By adjusting the preset angles of the flexible section, the off-axis and/or off-plane angles of the tip assembly the catheter can be adapted to ablate and/or map most if not all convex regions in the right atrium. Accordingly, improved focal and linear ablation and mapping can be accomplished with the catheter of the present invention despite convex contour or uneven tissue surface.

In yet another embodiment, the present invention is directed to a catheter configured for mapping and ablation a generally concave or tubular region of the heart, such as the cavity of the RA, RV, LA, LV, IVC or SVC or other tubular structures. In a detailed embodiment, the catheter has an intermediate section and a tip assembly adapted for mapping and/or ablation that is attached to the intermediate section by a pre-shaped flexible section that allows the tip assembly to be moved generally independently of the intermediate section. In a more detailed embodiment, the catheter comprises an elongated flexible tubular catheter body having proximal and distal ends. The intermediate section is mounted on the distal end of the tubular body and deflected with a curvature that approximates the generally concave contour of the cavitary or tubular structure. The tip assembly is attached to the end of the intermediate section by the flexible section which is configured with preset angles to extend the tip assembly off-axis and/or off-plane from the intermediate section so that the tip assembly can make suitable contact with the tissue surface of the cavitary or tubular structure

When deflected, the intermediate section of the catheter is configured to conform to the generally concave or tubular region so that motion of the heart is transferred to the catheter thereby providing stability to the tip assembly. The preshaped flexible section improves the ability of the tip assembly to access, contact and remain in contact with surrounding tissues of variable contour without undue pressure. Moreover, the preshaped flexible section may be reinforced to provide the tip assembly with stability in a selected angle. Accordingly, the catheter of the present invention has improved safety features and improved ablation and mapping capabilities.

In another embodiment, of the concave design the tip assembly is configured as an ablation assembly that may be irrigated, comprising a plurality of irrigation ports in between which an ablation coil electrode is wound. A porous covering, preferably made of expanded polytetrafluoroethylene, covers the coil electrode and irrigation ports. Fluid passes through the irrigation ports to the porous covering, which then disperses the fluid around the ablation assembly. This irrigation generally enables the creation of deeper lesions. In use, the distal end of the catheter is inserted into a patient's body and advanced atraumatically into cavity or tubular structure. The intermediate section is deflected onto or near a generally concave structure such as the RA, RV, LA, LV, SVC or IVC or other cavitary or tubular structures. The off-axis angle of the tip assembly readily allows the tip assembly to contact the surface notwithstanding the awkward angle imposed on the catheter by surface irregularities.

As the user operates the catheter and maneuvers the tip assembly, the deflected intermediate section advantageously synchronizes the catheter and the tip assembly with the motion of the heart while the pre-shaped flexible section advantageously allows the tip assembly to flex from the preset angle(s) as needed in order to remain in contact with the tissue. In one embodiment, as the tip assembly encounters protrusions and recesses while being dragged along the tissue surface, the tip assembly is jarred from its preset off axis angle but the flexible section allows the tip assembly to conform and ride along on the uneven surface without displacing the intermediate section.

By adjusting the preset angles of the flexible section, the off-axis and/or off-plane angles of the tip assembly the catheter can be adapted to ablate and/or map most if not all concave regions. Accordingly, improved focal and linear ablation and mapping can be accomplished with the catheter of the present invention despite concave contour or uneven tissue surface.

### BRIEF DESCRIPTION OF THE DRAWINGS

These and other features and advantages of the present invention will be better understood by reference to the following detailed description when considered in conjunction with the accompanying drawings, wherein:

FIG. 1 is an elevated side view of one embodiment of the catheter according to the invention where the flexible section is pre-shaped at an angle in the same direction as the deflection of the intermediate section;

FIG. 1A is a schematic perspective view of the distal end of the intermediate section, the flexible section and the tip assembly of the catheter of FIG. 1 in use at or near a generally convex region of the right atrium, such as a cava-tricuspid isthmus;

FIG. 2 is an elevated side view of another embodiment of the catheter according to the invention where the flexible section is preshaped at an angle opposite to the direction of deflection of the intermediate section;

FIG. 2a is a side cross-sectional view of a catheter body according to the catheter of FIG. 1, including the junction between the catheter body and the intermediate section;

FIG. 2b is a side cross sectional view taken of the side opposite that of FIG. 2a of the catheter body of FIG. 2a, including the junction between the catheter body and the intermediate section;

FIG. 3 is a side cross-sectional view of the intermediate section of the catheter of FIG. 1, including the junction between the intermediate section and the flexible section;

FIG. 3a is a longitudinal cross-sectional view of the intermediate section of FIG. 3 taken along line 3a-3a;

FIG. 3b is a side cross-sectional view of the flexible section of the catheter of FIG. 1, including the junction between the flexible section and the tip assembly;

FIG. 3c is a longitudinal cross-section view of the flexible section of FIG. 3 taken along line 3d-3d;

FIG. 4 is an enlarged side view of the distal end of the intermediate section, the flexible section and the tip assembly according to the embodiment of FIG. 1;

FIG. 5 is a top view of the intermediate section, the flexible section and the tip assembly of another embodiment of the catheter of the present invention, with the flexible section preset to support the tip assembly off-plane with the intermediate section;

FIG. 5A is a schematic perspective view of the intermediate section, the flexible section and the tip assembly of the catheter of FIG. 5 in use at or near a generally convex region of the right atrium, such as a Bundle of His;

FIG. 5b is a schematic perspective view of the distal end of the intermediate section, the flexible section and the tip assembly of the catheter of FIG. 2 in use at or near a generally concave region such as the RA, RV, LA, LV;

FIG. 5c is a schematic perspective view of the distal end of the intermediate section, the flexible section and the tip assembly of the catheter of FIG. 2 in use at or near a generally tubular region such as the SVC or IVC;

FIG. 5d is a schematic perspective view of the distal end of the intermediate section, the flexible section and the tip assembly of the catheter of FIG. 1 in use at or near a generally concave region such as the RA, RV, LA LV;

FIG. 5e is a schematic perspective view of the distal end of the intermediate section, the flexible section and the tip assembly of the catheter of FIG. 1 in use at or near a generally tubular region such as the SVC or IVC;

FIG. 6a is a close-up side view of an embodiment of an irrigated ablation assembly; and

FIG. 6b is a close-up longitudinal cross-sectional view of the ablation assembly depicted in FIG. 6a taken along line 5b-5b;

FIG. 7 is a side view of an embodiment of a catheter body, a deflectable intermediate section, and a tip assembly connected by a flexible section that extends the tip assembly in an off-axis direction generally opposite to the direction of deflection;

FIG. 8 is a side view of an embodiment of a catheter body, an intermediate section with a deflectable proximal section and a generally linear distal section, and a tip assembly connected by a flexible section that extends the tip assembly in an off-axis direction generally in the same direction as the direction of deflection; and

FIG. 9 is a side view of an embodiment of a catheter body, an intermediate section with a deflectable proximal section and a curved distal section, and a tip assembly connected by a flexible section that extends the tip assembly in an off-axis direction generally opposite to the direction of deflection.

### DETAILED DESCRIPTION OF THE INVENTION

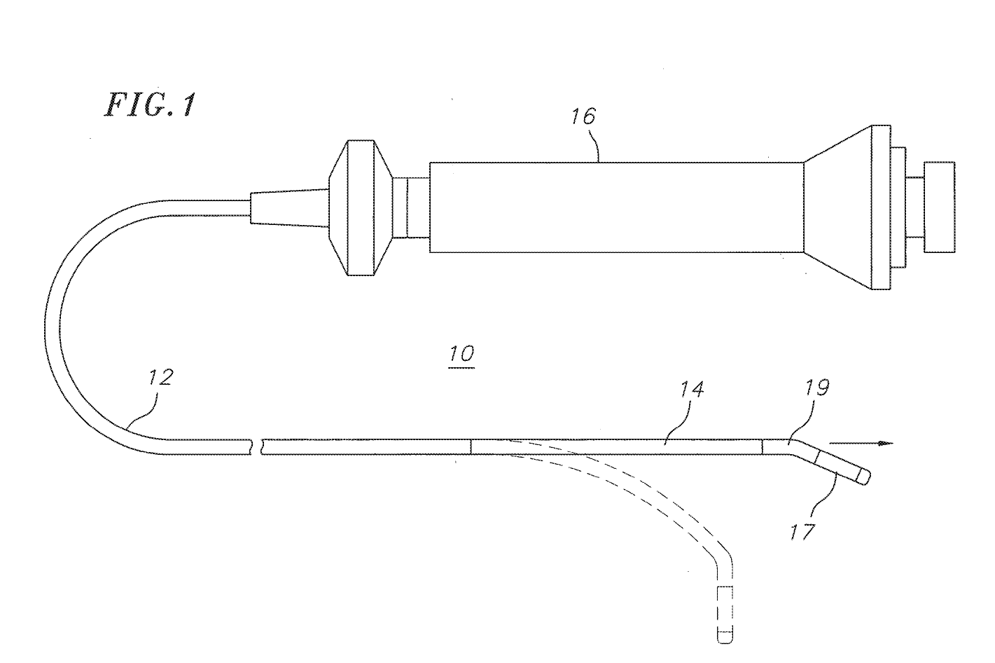

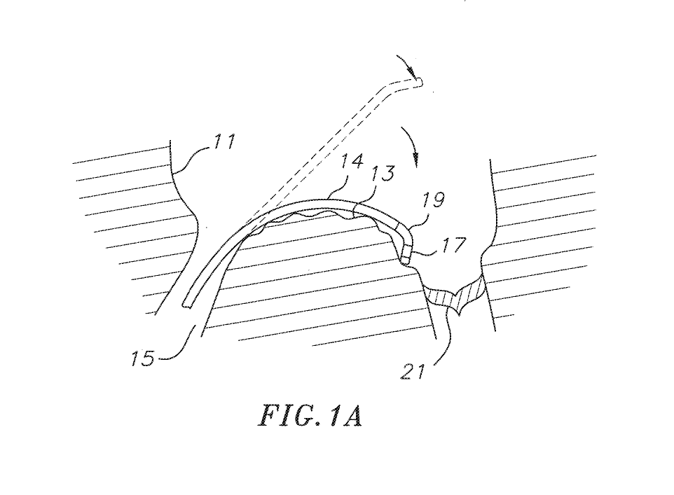

Referring to FIG. 1, the present invention provides a catheter 10 having a tip assembly 17 for mapping and/or ablation at its distal end. The catheter comprises an elongated catheter body 12 having proximal and distal ends, a deflectable intermediate section 14 at the distal end of the catheter body 12, and a control handle 16 at the proximal end of the catheter body. In accordance with a feature of the present invention, the tip assembly 17 is connected to the deflectable intermediate section 14 by a flexible section 19 which enables the tip assembly 17 to extend from the intermediate section 14 either in plane with or at a preset off-axis angle and/or off-plane angle. In the illustrated embodiment, the tip assembly 17 is adapted for ablation although it is understood by one of ordinary skill in the art that the tip assembly may be adapted for mapping applications, as well.

Referring to the embodiment of FIG. 1A, the catheter 10 is adapted for use in a right heart 11 to map or ablate a region with a generally convex contour such as an inferior vena cava-tricuspid isthmus 13. Advantageously, this region is accessible to the catheter 10 despite the catheter's entry to the atrium from an inferior vena cava 15 and the catheter's forward approach to the isthmus treatment site. In particular, the intermediate section 14 is deflected so the tip assembly can reach the isthmus despite the generally convex curvature of the isthmus. The deflection also enables the intermediate section to approximate and assume the convex curvature such that motion of the heart is transferred to catheter to stabilize the catheter. Moreover, notwithstanding the relatively acute and awkward angle of the isthmus encountered by the catheter extending from the inferior vena cava, contact between the tip assembly 17 and tissue surface of the isthmus 13 is enabled by an in-plane canting of the tip assembly in the direction of the deflection. The highly flexible section 19 connecting the tip assembly 17 and the intermediate section 14 not only enables the in-plane extension of the tip assembly but it also allows the tip assembly to maintain contact with the tissue surface despite the uneven and nonuniform surface of the isthmus 13 that spans between a tricuspid valve 21 and the inferior vena cava 15 which has recesses and protrusions that are encountered by the tip assembly 17 as it is dragged along to map and/or ablate the isthmus.

In accordance with a feature of the present invention, the flexible section 19 is preshaped with a configuration that attaches the tip assembly 17 of this embodiment at a predetermined off-axis angle relative to the intermediate section 14 in a direction of the deflection of the intermediate section. Moreover, the flexible section 19 has a bending modulus greater than that of the intermediate section 14 so the tip assembly 17 can flex and adjust to the contour of the isthmus tissue surface independently of the intermediate section 14. As shown in FIG. 1A, the off-axis extension of the tip assembly 17 from the intermediate section 14 enables contact between the tip assembly 17 and tissue surface of the isthmus. The ability of the tip assembly to flex and adjust permits the tip assembly 17 to contact tissue in recessed areas without exerting excess contact pressure in elevated areas reducing the risk of perforation.

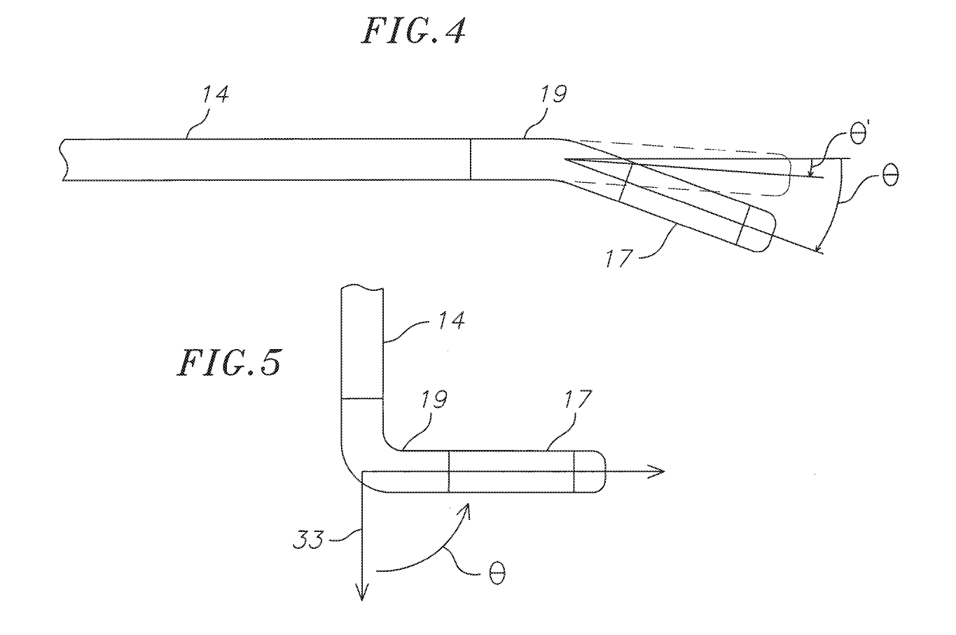

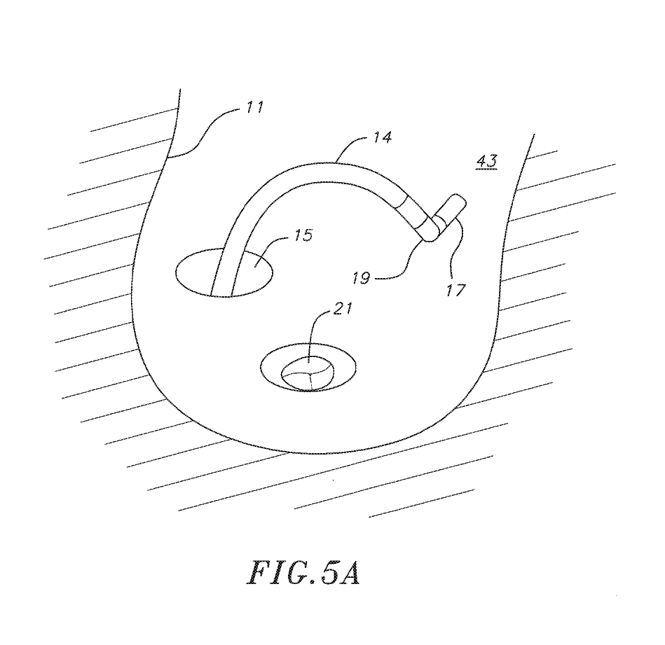

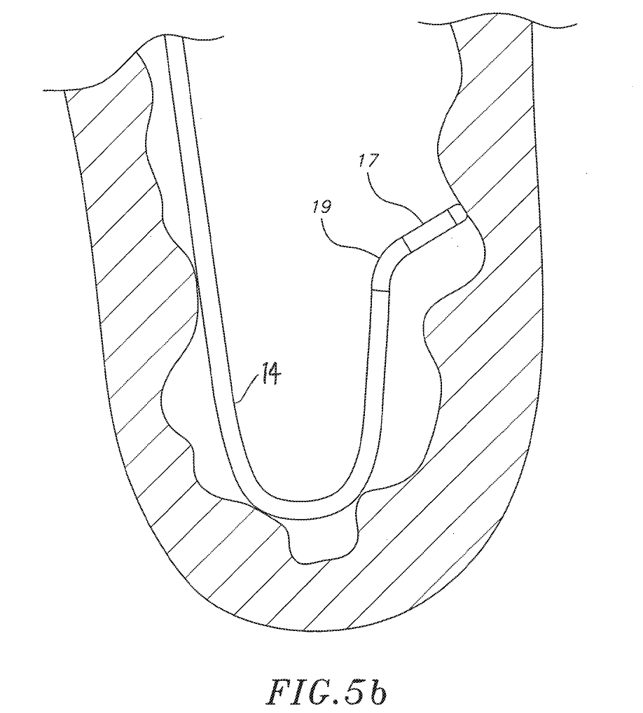

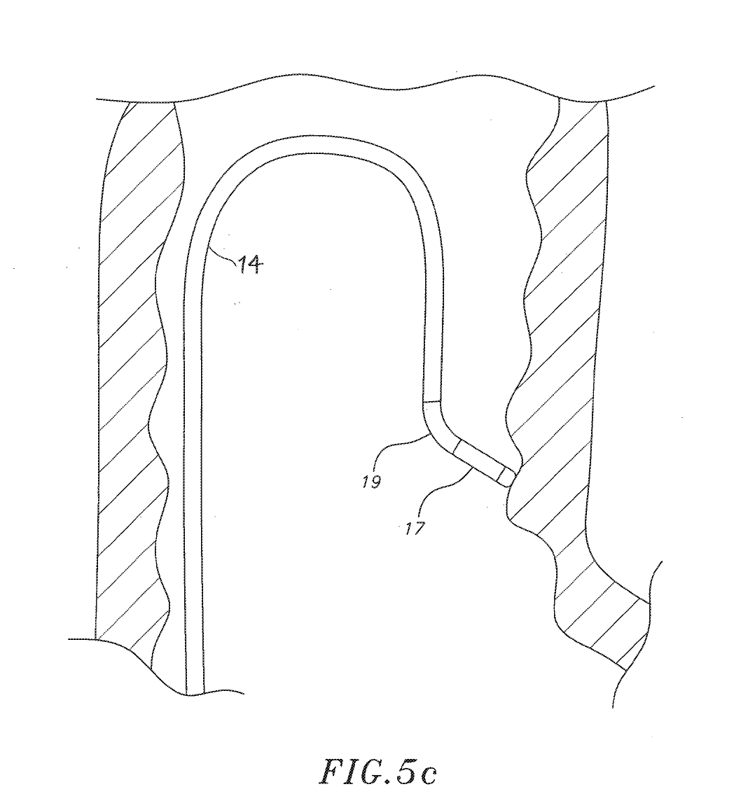

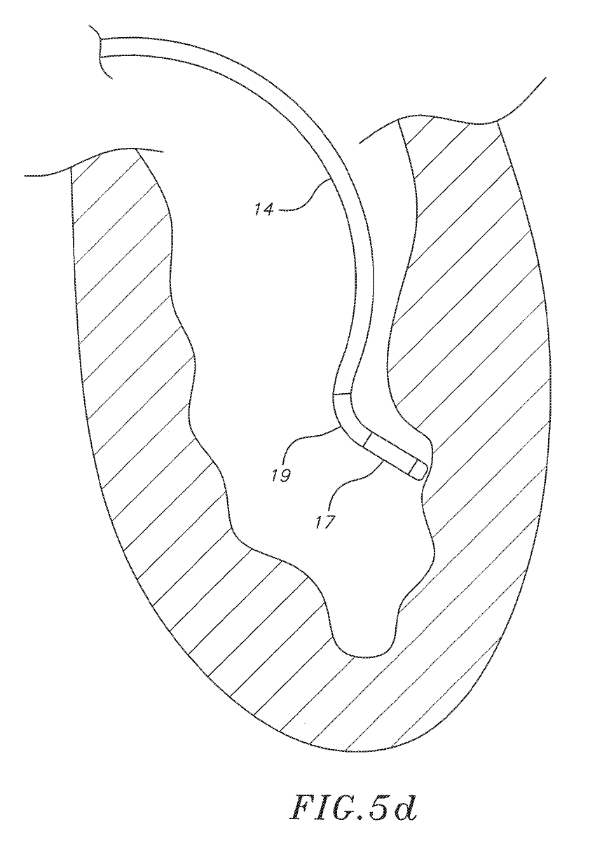

In the embodiment illustrated in FIG. 1, the catheter design is adapted for ablation of cavitary or tubular structures according to the method of introduction into the body as illustrated in FIGS. 5d and 5e although it is understood by one of ordinary skill in the art that the tip assembly may be adapted for mapping applications, as well.

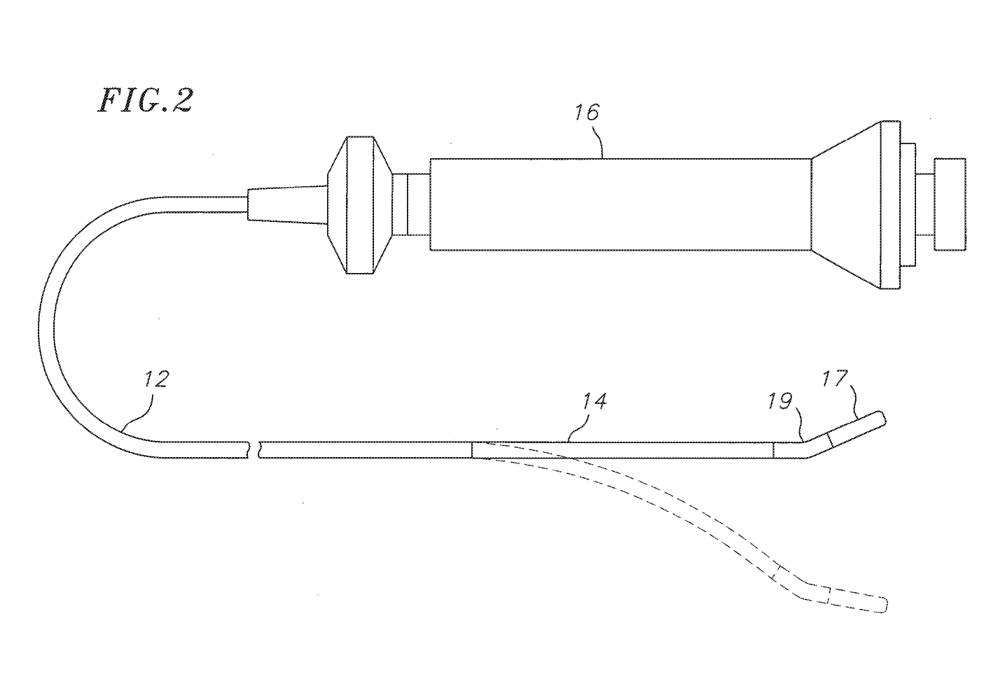

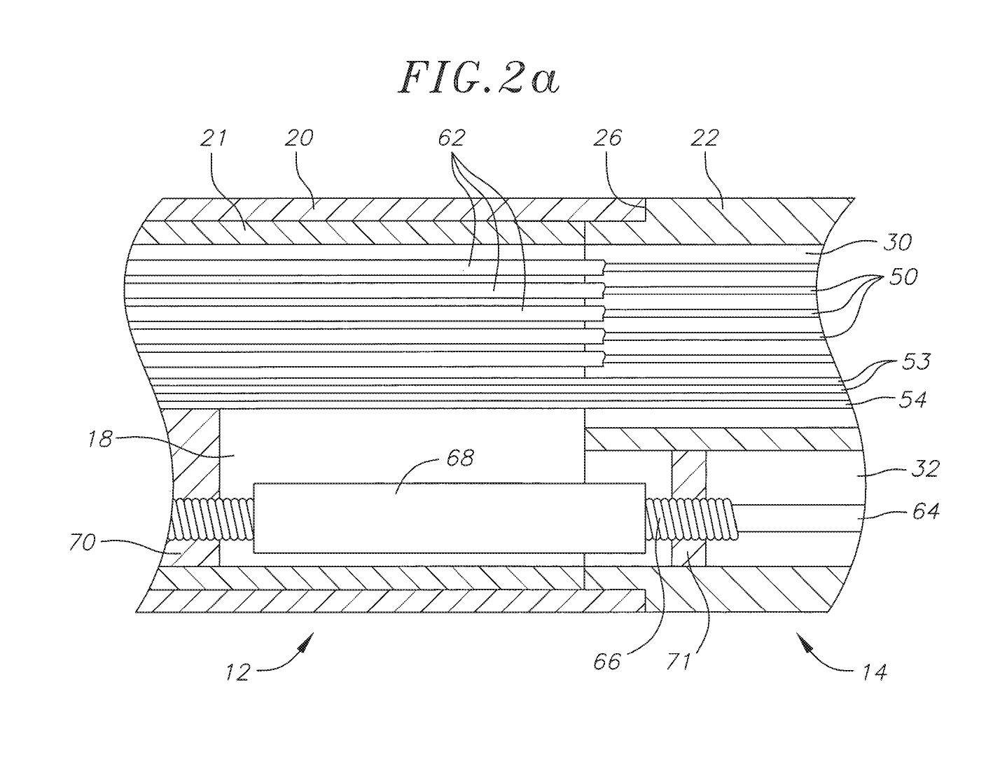

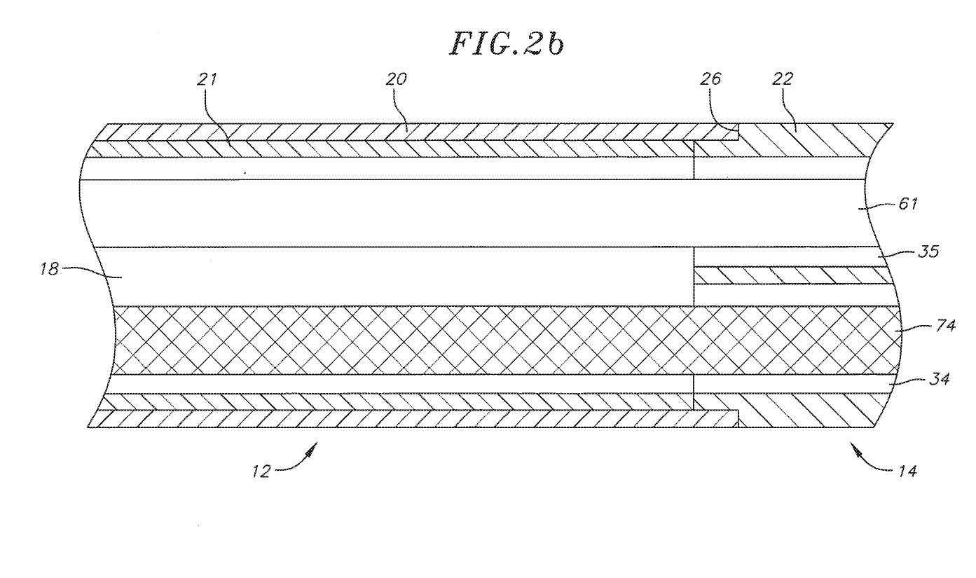

Referring to FIG. 2, the present invention also provides a catheter 10 having a tip assembly 17 for mapping and/or ablation at its distal end. The catheter comprises an elongated catheter body 12 having proximal and distal ends, a deflectable intermediate section 14 at the distal end of the catheter body 12, and a control handle 16 at the proximal end of the catheter body. In accordance with a feature of the present invention, the tip assembly 17 is connected to the deflectable intermediate section 14 by a flexible section 19 which enables the tip assembly 17 to extend from the intermediate section 14 either in plane with or at a preset off-axis angle and/or off-plane angle.

In the illustrated embodiments of FIGS. 5b and 5c, the tip assembly 17 is adapted for ablation of cavitary or tubular structures according to the method of introduction into the body, although it is understood by one of ordinary skill in the art that the tip assembly may be adapted for mapping applications, as well.

With reference to FIGS. 2a and 2b, the catheter body 12 comprises an elongated tubular construction having a single, axial or central lumen 18. The catheter body 12 is flexible, i.e., bendable, but substantially non-compressible along its length. The catheter body 12 can be of any suitable construction and made of any suitable material. A presently preferred construction comprises an outer wall 20 made of polyurethane or PEBAX. The outer wall 20 comprises an embedded braided mesh of stainless steel or the like to increase torsional stiffness of the catheter body 12 so that, when the control handle 16 is rotated, the intermediate section 14 of the catheter 10 is able to rotate in a corresponding manner.

The outer diameter of the catheter body 12 is not critical, but is preferably no more than about 9 french, more preferably about 7 french. Likewise, the thickness of the outer wall 20 is not critical, but is thin enough so that the central lumen 18 can accommodate a puller wire, one or more lead wires, and any other desired wires, cables or tubes. If desired, the inner surface of the outer wall 20 is lined with a stiffening tube 21 to provide improved torsional stability. A particularly preferred catheter 10 has an outer wall 20 with an outer diameter of from about 0.090 inches to about 0.094 inches and an inner diameter of from about 0.061 inches to about 0.065 inches.

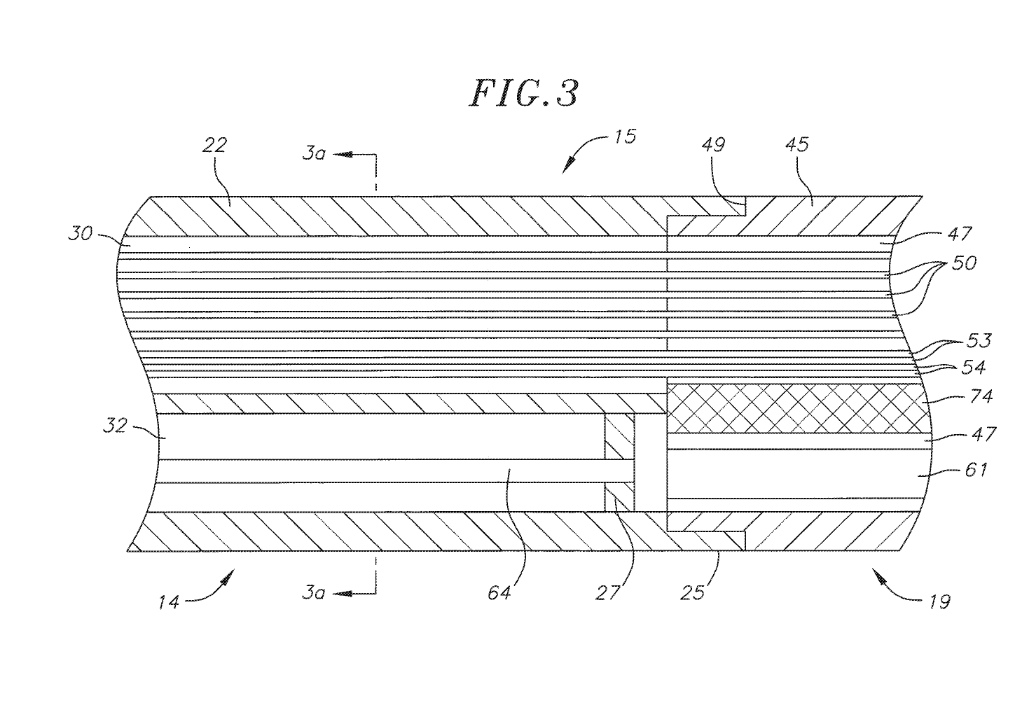

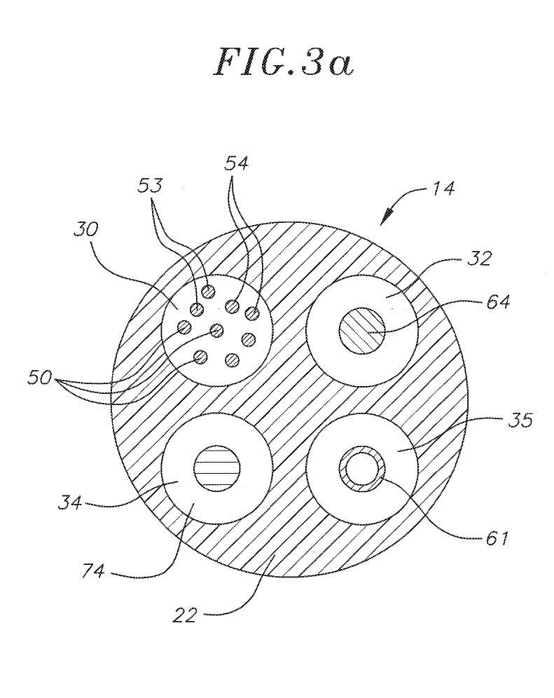

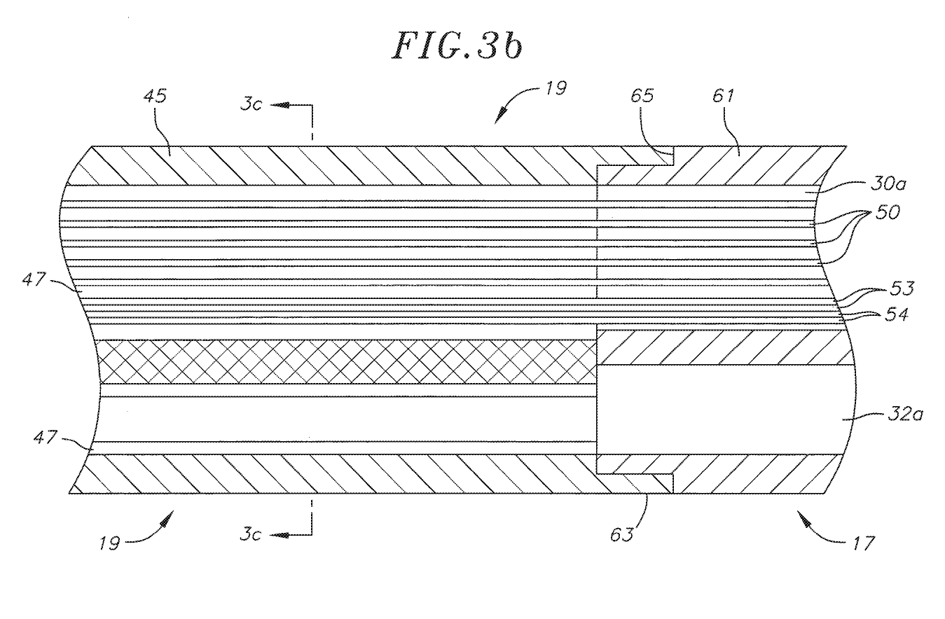

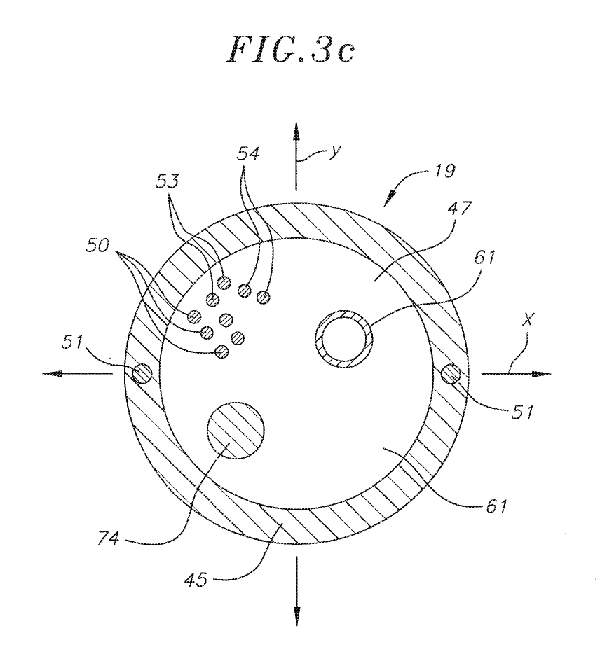

The intermediate section 14 comprises a short section of tubing 22 having multiple lumens, as shown in FIG. 3a. In one embodiment, a first lumen 30 carries one or more lead wires 50 and any other components (e.g., thermocouple wires 53 and 54 for monitoring tissue temperature) extending along the catheter (FIGS. 2a, and 3). A second lumen 32 carries a puller wire 64 (FIG. 3). As also shown in FIG. 2b, a third lumen 34 carries an electromagnetic sensor cable 74, and a fourth lumen 35 carries an irrigation tube 61 for supplying fluid to the tip assembly 17. The tubing 22 is made of a suitable non-toxic material that is preferably more flexible than the catheter body 12. A presently preferred material for the tubing 22 is braided polyurethane, i.e., polyurethane with an embedded mesh of braided stainless steel or the like. The number of lumens or the size of each lumen is not critical, but is sufficient to house the lead wires, puller wire, electromagnetic sensor cable, thermal sensors and/or irrigation tube(s) depending on the embodiment.

The useful length of the catheter 10, i.e., that portion that can be inserted into the body excluding the tip assembly 17, can vary as desired. Preferably the useful length ranges from about 110 cm to about 120 cm. The length of the intermediate section 14 is a relatively small portion of the useful length, and preferably ranges from about 3.5 cm to about 10 cm, more preferably from about 5 cm to about 6.5 cm.

A preferred means for attaching the catheter body 12 and the intermediate section 14 is illustrated in FIGS. 2a and 2b. The proximal end of the intermediate section 14 comprises an outer circumferential notch 26 that receives the inner surface of the outer wall 20 of the catheter body 12. The intermediate section 14 and catheter body 12 are attached by glue or the like.

If desired, a spacer (not shown) can be located within the catheter body between the distal end of the stiffening tube 21 and the proximal end of the intermediate section 14. The spacer provides a transition in flexibility at the junction of the catheter body 12 and intermediate section 14, which allows the junction to bend smoothly without folding or kinking. A catheter having such a spacer is described in U.S. Pat. No. 5,964,757, the entire disclosure of which is incorporated herein by reference.

As shown in FIG. 2a, the puller wire 64 is provided for deflection of the intermediate section 14 (see FIG. 1A). The puller wire 64 extends through the catheter body 12. Its proximal end is anchored to the control handle 16, and its distal end is anchored to the distal end of the intermediate section 14 in the lumen 32 by any suitable means, for example, adhesives forming glue joint 27 (FIG. 3). The puller wire 64 is made of any suitable metal, such as stainless steel or Nitinol, and is preferably coated with Teflon® or the like. The coating imparts lubricity to the puller wire 64. The puller wire 64 preferably has a diameter ranging from about 0.006 to about 0.010 inch.

A compression coil 66 is situated within the catheter body 12 in surrounding relation to the puller wire 64, as shown in FIG. 2a. The compression coil 66 extends from the proximal end of the catheter body 12 to the proximal end of the intermediate section 14. The compression coil 66 is made of any suitable metal, preferably stainless steel. The compression coil 66 is tightly wound on itself to provide flexibility, i.e., bending, but to resist compression. The inner diameter of the compression coil 66 is preferably slightly larger than the diameter of the puller wire 64. The Teflon® coating on the puller wire 64 allows it to slide freely within the compression coil 66. The outer surface of the compression coil 66 is covered by a flexible, non-conductive sheath 68, e.g., made of polyimide tubing.

The compression coil 66 is anchored to the outer wall of the catheter body 12 by proximal glue joint 70 and at its distal end to the intermediate section 14 by distal glue joint 71. Both glue joints 70 and 71 preferably comprise polyurethane glue or the like. The glue may be applied by means of a syringe or the like through a hole made between the outer surface of the catheter body 12 and the central lumen 18. Such a hole may be formed, for example, by a needle or the like that punctures the outer wall 20 of the catheter body 12 which is heated sufficiently to form a permanent hole. The glue is then introduced through the hole to the outer surface of the compression coil 66 and wicks around the outer circumference to form a glue joint about the entire circumference of the compression coil.

Longitudinal movement of the puller wire 64 relative to the catheter body 12, which results in deflection of the intermediate section 14, is accomplished by suitable manipulation of the control handle 16. Examples of suitable control handles for use in the present invention are disclosed in U.S. Pat. Nos. Re 34,502 and 5,897,529, the entire disclosures of which are incorporated herein by reference. As mentioned, deflection of the intermediate section 14 by longitudinal movement of the puller wire 64 allows the intermediate section 14 to generally approximate and conform to the convex curvature of the isthmus. As such, the deflected intermediate section 14 can sit on the isthmus and transmit the motion of the heart during systole, diastole and respiration to the entire catheter. The distal tip of the catheter is thus both stable and moves in synchrony with the heart. This allows the tip assembly of the catheter to conform to irregularities without undue pressure reducing the risk of any of the following: a) direct mechanical perforation because the flexible section readily flexes so as to reduce the maximal tip pressure that can be applied by the proximal portion of the catheter, b) perforation due to steam pop, as the flexible section allows the tip assembly to be displaced off the surface allowing the steam to exit into the right atrium rather than the tip pressure forcing the steam into the myocardium and out into the pericardial space; c) impedance rise, excess temperature, thrombus and char formation, as the maximum tip pressure is limited by the flexible section reducing the likelihood of the tip assembly being buried in the tissue, reducing cooling by the circulating blood.

In accordance with another feature of the present invention, the tip assembly 17 is attached to the intermediate section 14 by the pre-shaped flexible section 19. As shown in FIG. 4, the flexible section 19 supports the tip assembly 17 at an in-plane off-axis angle from the distal end of the intermediate section 14. Using an angle θ to define the off-axis angle, the angle θ may range between about 0 degrees to about 90 degrees, preferably between about 10 degrees to 60 degrees, and more preferably about 30 degrees. With the tip assembly 17 in plane but canted off axis in the direction of deflection of the intermediate section 14, the angle θ effectively increases the deflection angle to enable the tip assembly 17 to reach further around the isthmus and contact the tissue surface. The flexibility of the section 19 allows the angle θ to be varied from the initially set angle to zero degree (or on-axis position) with minimal force applied to the tip assembly 17 through contact with the tissue.

The flexible section 19 is constructed with sufficient shape memory and/or sufficient flexibility and elasticity so that the tip assembly 17 can temporarily assume a different (greater or lesser) angle θ as needed for the tip assembly to pivot at its proximal end. The flexible section 19 can be sufficiently soft to allow the tip assembly 17 to be displaced from its preset off-axis angle θ to an on-axis angle where θ is about zero, and sufficiently elastic to return (or at least bias the return of) the tip assembly 17 to its preset off-axis angle θ thereafter, whether the displacement was caused by a formation 37 in the tissue surface, the tip assembly being caught or buried in the surrounding tissue, or a “steam pop” where a build up of pressure dislodges the tip assembly from tissue contact. To that end, the flexible section 19 has a relatively high flexural modulus measuring on a Durometer scale no greater than about 25 D to 35 D and/or no greater than about ½ to ¼ of the Durometer measurement of the intermediate section 14. The flexible section 19 acts as a “shock absorber” when the tip assembly is jarred or otherwise displaced from its preset position. The flexible section 19 enables the tip assembly 17 to pivot away from the recess 37 independently of the intermediate section 14 so that the tip assembly can remain in contact with the tissue. Referring to FIG. 4, as the catheter 10 is advanced, withdrawn or otherwise maneuvered around the isthmus, the tip assembly 17 can move from its preset angle θ (solid lines) to a displaced position at angle θ′ (broken lines) without significantly displacing the intermediate section 14 whether or not deflected.

As understood by one of ordinary skill in the art, the shape memory of the material 45 of the flexible section 19 also allows the catheter to be advanced atraumatically in the patient's body in a generally straight configuration through a vein or artery and yet be able to assume its preformed shape when it reaches the heart.

Referring to FIGS. 5 and 5A, the highly flexible section 19 in another embodiment may also be configured to support the tip assembly 17 off-plane from the intermediate section 14 at a variety of radial angles. As shown in FIG. 5A, the catheter 10 is adapted to map and/or ablate another region in the right atrium with a generally convex contour, such as the Bundle of His region 43 (or “His region” hereinafter), although the His region may pose a further challenge as the region is also slightly canted anteriorly from the inferior vena cava 15.

The His region 43 is accessible to the catheter 10 despite the catheter's entry to the atrium from the inferior vena cava 15 and the catheter's forward approach to the His region. As with the foregoing embodiment, the intermediate section 14 is deflected so the tip assembly 17 can reach the His region. Where the deflected intermediate section 14 can approximate and assume a convex curvature near the His region, motion of the heart is transferred to catheter to stabilize the catheter. In accordance with a feature of the present invention, contact between the tip assembly 17 and tissue surface of the His region 43 is enabled by an off-plane extension of the tip assembly 17 (which may or may not also extend at an off-axis angle from the intermediate section 14). The highly flexible section 19 between the tip assembly 17 and the intermediate section 14 allows the tip assembly to maintain contact with the His tissue surface despite the uneven and nonuniform surface of the His region which has recesses and protrusions that are encountered by the tip assembly 17 as it is dragged along to map and/or ablate the His region.

Referring to FIG. 5 (a top view of the tip assembly 17 and intermediate section 14), using angle γ to define the radial angle from plane of deflection 33 of the intermediate section 14, the angle γ may range between about 0 to 180 degrees, preferably about 20 to 90 degrees, and more preferably about 45 degrees as shown in the embodiment of FIG. 5 (compared with the embodiment of FIG. 4a where the angle γ is about zero degrees).

As illustrated in FIG. 5A, where the catheter enters the right atrium from the inferior vena cava, the off-plane radial angle γ of the tip assembly 17 extending from the deflected intermediate section 14 allows the tip assembly to reach in an angle generally lateral of the deflection direction. As such, the His region is readily accessed by the tip assembly 17 for mapping and/or ablation.

It is understood by one of ordinary skill in the art that the off axis angle θ and the off-plane angle γ may be preset independently of one another. That is, the catheter 10 of the present invention may have the tip assembly 17 extend from the intermediate section 14 at any combination of the angle θ and the angle γ in accordance with their respective ranges set forth above, as desired or appropriate. In one embodiment of the catheter 10 for use in ablating and/or mapping the His region, the angle θ is about 20 degrees and the angle γ is about 90 degrees.

As mentioned, the flexible section 19 allows the tip assembly to be displaced without displacing the intermediate section 14. In one embodiment, the tip assembly 17 can be displaced from its preset off-axis and/or off-plane angle under a force or weight of merely about 0.25 to about 2.0 oz, and more preferably about 1.0 ounce. As such, the flexible section 19 provides sufficient flexibility to reduce the risk of injury that can result from the tip assembly 17 inadvertently perforating tissue or being buried in the tissue and overheating. As understood by one of ordinary skill in the art, the force required to displace or capable of displacing the tip assembly from the preset angle(s) also depends on the point of application of the force to the tip assembly, as well as the length of the tip assembly.

The flexible section 19 comprises a short section of material 45 (e.g., tubing) with a central lumen 47 through which the lead wire(s) 50, thermocouple wires 53 and 54, sensor cable 74 and irrigation tube 61 extend distally and connect to the tip assembly 17. A junction 25 of the intermediate section 14 and the flexible section 19 is shown in FIG. 3. The proximal end of the material 45 of the tip assembly 17 comprises an outer circumferential notch 49 that receives the inner surface of the tubing 22 of the intermediate section 14. The intermediate section 14 and the flexible section 19 are attached by glue or the like. The flexible section can be made of polyurethane, PEBAX, silicone or combinations thereof and is preformed (used generally interchangeably with “preshaped” herein) with shape memory by placing the tubing 45 in a delrin mold and heating the mold at about 100° C. for about 30 minutes. Alternatively, the tip assembly and the flexible section may be formed as a single unit with the flexibility of the tip assembly or flexible section determined by the incorporated sensors, wires and electrodes. The length of the flexible section 19 can vary as desired and can range between about 0.1 cm and 2.0 cm, preferably between about 0.2 cm and 1.0 cm, and more preferably about 5.0 cm.

Moreover, where desirable or appropriate, lateral stability can be provided in the tip assembly 17 with the use of struts or ribbons 51 provided in walls of the material 45 of the flexible section 19, as shown in FIG. 3c, or elsewhere on or in the tubing as desirable. A pair of struts 51 can be aligned along any diameter of the material 45 to stabilize the tip assembly. In the embodiment of FIG. 3c, the struts minimize lateral movement along direction X but still allow displacement along direction Y.

Recognizing that atria and isthmuses can come in different shapes and sizes, the intermediate section 14 may have a length ranging between about 1.0 cm and 20 cm, preferably between about 4.0 cm and 16 cm, and more preferably between about 7.0 cm and 12 cm. The intermediate section 14 may assume a “J” curve when deflected for flutter treatment and procedures and a “D” curve for HIS treatment and procedures. However, it is understood that the intermediate section and its deflection curvature may assume a variety of sizes and shapes as desirable or appropriate for the intended region of ablation or mapping.

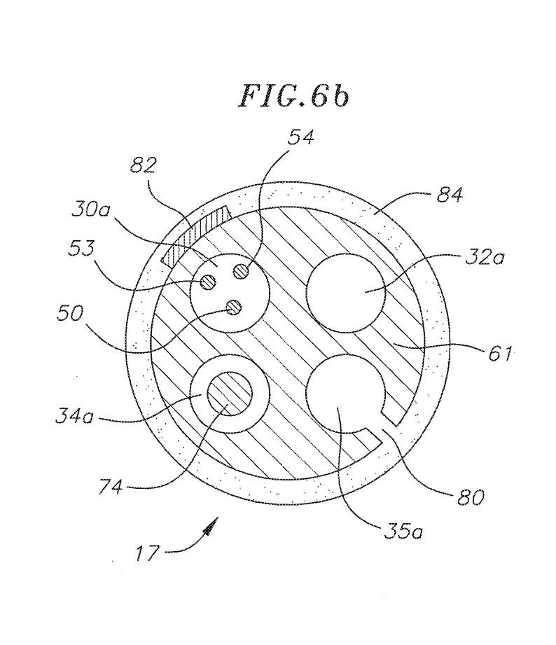

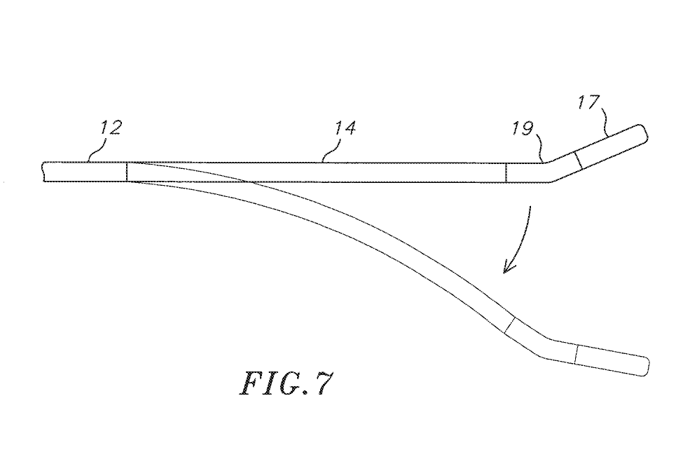

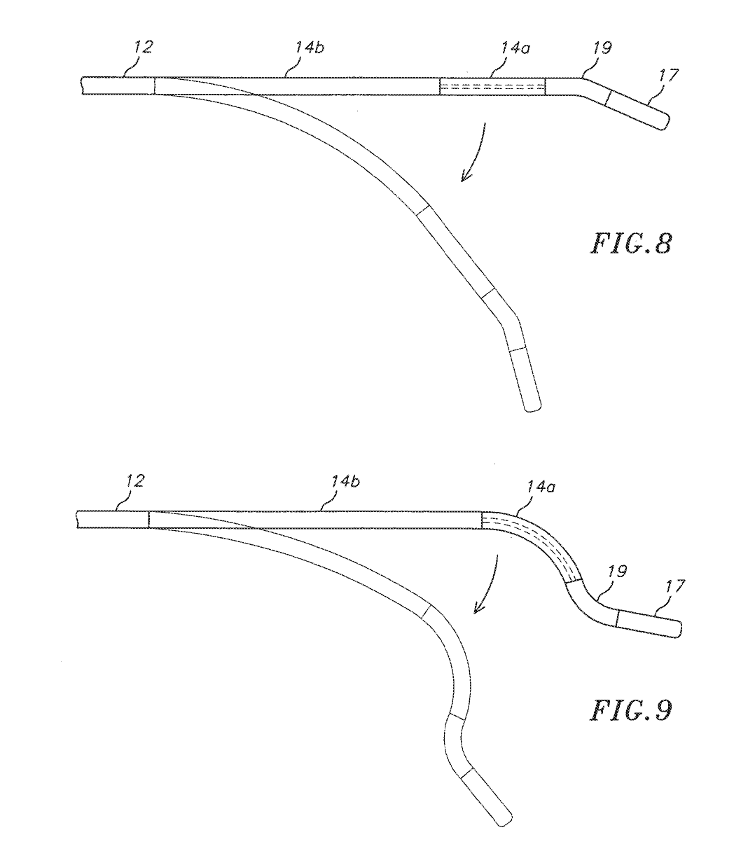

In addition, as shown in FIGS. 7, 8 and 9, it is understood by one of ordinary skill in the art that the flexible section 19 may flexibly extend the tip assembly 17 in a direction generally opposite to the direction of deflection of the intermediate section 14 (FIG. 7), or that the intermediate section may be divided into distal and proximal sections 14a and 14b with the proximal intermediate section 14b deflectable and the distal intermediate section 14a with shape-memory configured generally straight (FIG. 8) or with a curve (FIG. 9), as desirable or appropriate for the intended region of ablation or mapping.

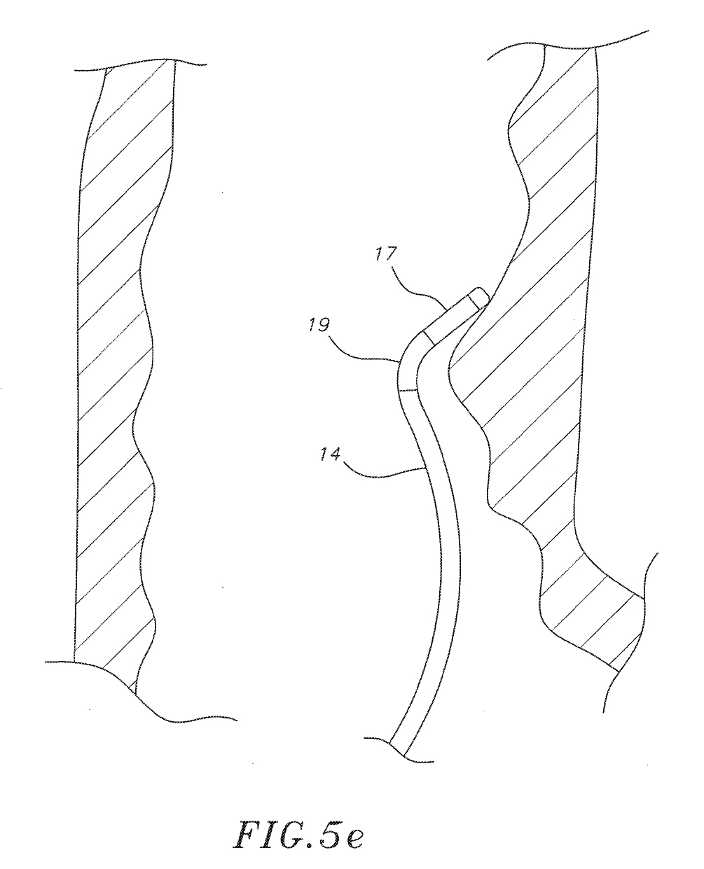

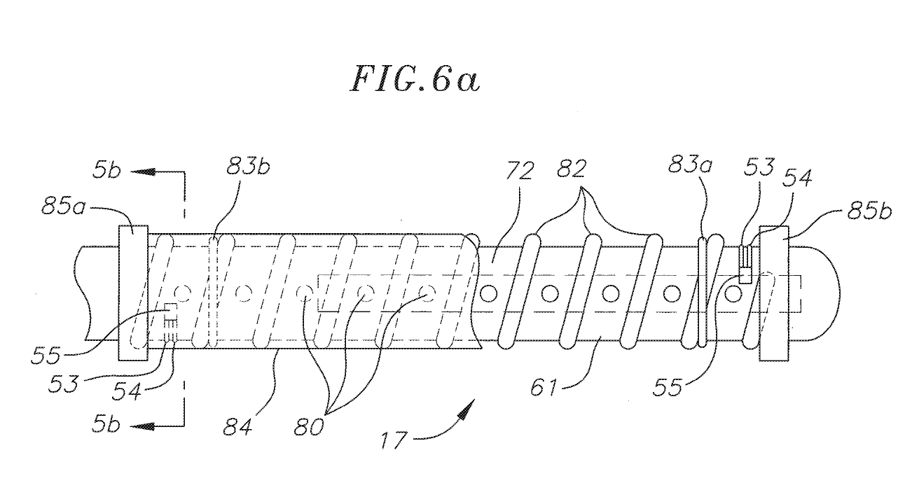

In illustrated embodiment, the tip assembly 17 comprises a short section of material tubing 61 (e.g., tubing) (FIGS. 3b, 6b) comprising four lumens 30a, 32a, 34a and 35a, generally corresponding to and aligned with the four lumens 30, 32, 34 and 35 respectively, of the intermediate section 14. The length of the tip assembly 17 can be varied as desired, but preferably ranges between about 8 mm to about 15 mm, and more preferably is about 10 mm. A junction 63 of the flexible section 19 and the tip assembly 17 is shown in FIG. 3b. The proximal end of the material 61 of the tip assembly 17 comprises an outer circumferential notch 65 that receives the inner surface of the tubing 45 of the flexible section 19. The flexible section 19 and tip assembly 17 are attached by glue or the like.

FIG. 6a illustrates an embodiment of the tip assembly 17 configured as an ablation assembly. A coil electrode 82 is coiled around the length of the ablation assembly 17. The longitudinal span of the coil electrode 82 may be made of any suitable metal, preferably platinum/iridium and ranges in length from about 6 to about 10 mm, preferably about 8 mm to generally match the length of the ablation assembly 17.

In the disclosed embodiment, the ablation assembly 17 is irrigated and comprises a plurality of irrigation ports 80 disposed along most of the length of the ablation assembly 17 through which fluid can pass to the outer surface of the ablation assembly to cool the ablation site. In the illustrated embodiment, the coil and the irrigation ports 80 are arranged so that an irrigation port lies between each wind of the coil electrode 82. The irrigation ports may comprise round holes formed on the surface of the tubing 61 on the side of the ablation assembly 17 in communication with the fourth lumen 35A which is supplied fluid by the irrigation tube 61 whose distal end is slightly proximal of the most proximal irrigation port. Any number of irrigation ports 80 may be used. In the illustrated embodiment, the tubing 61 of the ablation assembly 17 is configured with about 10 irrigation ports 80. The circumference of each round hole can measure about 20/1000 inch. As shown in FIGS. 6a and 6b, a porous protective covering 84, of, for example, expanded polytetrafluoroethylene (EPTFE), is disposed over the tubing 61 in surrounding relation to and covering the coil electrode 82 and irrigation ports 80

A tip electrode lead wire 50 (FIG. 6b) connects the coil electrode 82 to a suitable source of ablation energy (not shown), preferably radio frequency (RF) energy. The distal end of the lead wire 50 is attached to the proximal end of the coil electrode 82. The proximal end of the lead wire 50 is electrically connected to the source of ablation energy as is known in the art. The lead wire 50 extends through the first lumen 30a of the ablation assembly 17, the central lumen 47 of the flexible section 19, the first lumen 30 of the intermediate section 14, the central lumen 18 of the catheter body 12, and the control handle 16, and terminates at its proximal end in a connector (not shown).

As shown in FIG. 6a, if desired, mapping and/or ablation ring electrodes 83a and 83b may be mounted on the ablation assembly 17. Additional ring electrodes may be contained within the ablation assembly or the intermediate section depending on spacing or the application of the catheter. The ring electrodes 83a and 83b can be mounted over the coil electrode 82 and underneath the porous covering 84. In the illustrated embodiment, the first ring electrode 83a is positioned in between the two distal most irrigation ports 80. The second ring electrode 83b is positioned in between the two proximal most irrigation ports 80. The ring electrodes 83a and 83b are mounted to the coil electrode 82 by any suitable means, for example by welding, soldering or the like. As such, the ring electrodes 83a and 83b are electrically connected to the coil electrode 82 and its associated lead wire for ablation purposes. The ring electrodes 83a and 83b serve in part to hold the coil electrode 82 in place on the tubing 61 of the ablation assembly. The ring electrodes 83a and 83b also serve to flatten the coil electrode 82 on the surface of the tubing 61, thereby preventing any rough edges of the coil electrode 82 from cutting into the porous covering 84.

Any conventional temperature sensors, e.g. thermocouples or thermistors, may be used. In the embodiment shown in FIGS. 2a, 3 and 6a, the temperature sensors comprise two thermocouples formed by two enameled wire pairs. One wire of each wire pair is a copper wire 53, e.g., a number “40” copper wire. The other wire of each wire pair is a constantan wire 54. The wires 53 and 54 of each wire pair are electrically isolated from each other except at their distal ends where they are twisted together, covered with a short piece of plastic tubing 55 (FIG. 6a), e.g., polyimide, and covered with epoxy. The wires 53 and 54 of each wire pair extend out a hole in the side wall of the tubing 61 and are anchored to the outer surface of tubing 61. The hole in the side wall of the distal region is sealed by a plug. Any suitable seal may be used, for example glue or the like. Each plastic tubing 55 is mounted on the outer surface of the tubing 61 by polyurethane glue or the like. One of the two thermocouples is anchored immediately distal the distal most irrigation port 80, as shown in FIG. 6a. The second of the two thermocouples is anchored immediately proximal the proximal most irrigation port 80. The wires 53 and 54 extend through the first lumen 30 in the ablation assembly 17 and intermediate section 14, through the central lumen 18 of the catheter body 12 and out through the control handle 16 to a connector (not shown) connectable to a temperature monitor (not shown).

Additional electrodes may be incorporated depending on the application electrode width and spacing, as well as the preferences of the operator of the catheter. If desired, one or more mapping and/or ablation ring electrodes can be mounted on the tubing 45 of the flexible section 19 and tubing 61 of the ablation assembly 17, as shown in FIGS. 5 and 6a. These ring electrodes might be desirable, for example, for mapping the region to be ablated before ablation begins or after ablation to assure that the lesions blocked the electrical activity as desired. A ring electrode 85A can be mounted on the proximal end of the tubing 61 of the ablation assembly 17 over the porous covering 84 so that the proximal end of the porous covering 84 can be tucked underneath the ring electrode 85A to lock the proximal position of the porous covering 84. Also, a second ring electrode 85b can be mounted on the distal end of the tubing 61 so that the distal end of the porous covering 84 can be tucked underneath the ring electrode 85b to lock the distal position of the porous covering 84.

In other embodiment, the tip assembly 17 whether adapted for mapping or ablation may be constructed with or without irrigation, with or without temperature sensors, using suitable ring electrodes for sensing and/or ablation, as understood by one of ordinary skill in the art. The relationship between the tip assembly and the flexible section remains generally as described herein.

In addition, as better shown in FIGS. 4 and 4A, two additional ring electrodes 86a and 86b for mapping are mounted on the flexible section 19. The first ring electrode 86a is positioned approximately 5 mm proximal the proximal locking ring electrode 85A and is used to confirm the position of the ablation assembly in the atrium. The second ring electrode 86b is positioned approximately 2.5 mm proximal the first ring electrode 86a and is also used to confirm the position of the ablation assembly in the atrium. As understood by one of ordinary skill in the art, the mapping electrodes may be mounted at different locations on the ablation assembly 17, flexible section 19 and/or intermediate section 14 as desired.

In FIG. 3, each ring electrode 85A, 85b, 86a, 86b and 86c is connected to a corresponding lead wire 50. The distal end of each lead wire 50 is attached to the corresponding ring electrode. The proximal end of each lead wire 50 is electrically connected to a suitable monitoring device for monitoring electrical activity. Each lead wire 50 extends through the first lumen 30a of the ablation assembly 17, the central lumen 47 of the tubing 45, the first lumen 30 of the intermediate section 14, the central lumen 18 of the catheter body 12, and the control handle 16, and terminates at its proximal end in a connector (not shown).

As shown in FIG. 2a, the portion of each lead wire 50 extending through the control handle 16, the central lumen 18 of the catheter body 12, and at least the proximal section of the intermediate section 14 is enclosed within a protective sheath 62 to prevent contact with other lead wires or other components of the catheter. The protective sheath 62 can be made of any suitable material, preferably polyimide. The protective sheath 62 is anchored at its distal end to the proximal end of the intermediate section 14 by gluing it in the first lumen 30 with polyurethane glue or the like. As would be recognized by one skilled in the art, the protective sheath 62 can be eliminated if desired.

As shown in FIG. 6a, an electromagnetic navigation sensor 72 may be contained within the ablation assembly 17. The electromagnetic sensor 72 is preferably situated at the distal tip of the ablation assembly 17 and is approximately 5 mm long. The electromagnetic sensor 72 is positioned in the third lumen 34a of the ablation assembly 17. The electromagnetic sensor 72 is mounted to the tubing 61 of the ablation assembly 17 by any suitable means, e.g. by polyurethane glue or the like.

The electromagnetic sensor 72 is connected to an electromagnetic sensor cable 74, which extends through the third lumen 34a in the ablation assembly 17, the central lumen 47 of the flexible section 19, the third lumen 34 of the intermediate section 14, through the catheter body 12, and out through the control handle 16. The electromagnetic sensor cable 74 comprises multiple wires encased within a plastic covered sheath. In the control handle 16, the sensor cable 74 is connected to a circuit board (not shown). The circuit board amplifies the signal received from the electromagnetic sensor 72 and transmits it to a computer in a form understandable by the computer. Because the catheter is designed for a single use only, the circuit board may contain an EPROM chip which shuts down the circuit board approximately 24 hours after the catheter has been used. This prevents the catheter, or at least the electromagnetic sensor from being used twice.

Suitable electromagnetic sensors for use with the present invention are described, for example, in U.S. Pat. Nos. 5,558,091, 5,443,489, 5,480,422, 5,546,951, and 5,391,199, the disclosures of which are incorporated herein by reference. A preferred electromagnetic sensor 72 has a length of from about 6 mm to about 7 mm, preferably about 5 mm, and a diameter of about 1.3 mm.

In FIG. 3a, the irrigation tube 61 may be made of any suitable material, and is preferably made of polyimide tubing. A preferred irrigation tube has an outer diameter of from about 0.032 inch to about 0.036 inch, and an inner diameter of from about 0.028 inch to about 0.032 inch. The irrigation tube 61 extends through the central lumen 18 of the catheter body 12 (FIG. 2b), the fourth lumen 35 of the intermediate section 14, the central lumen 47 of the flexible section 19, and the fourth lumen 35A of the ablation assembly 17 (FIG. 3a), and terminates slight proximal of the most proximal irrigation port 80 in the ablation assembly 17. The proximal end of the irrigation tube 61 extends through the control handle 16 and terminates in a luer hub or the like (not shown). Fluid is introduced into the irrigation tube 61 through the luer hub. The fluid, e.g. saline, is then introduced to the fourth lumen 35A of the ablation assembly 17 by the irrigation tube 61 and passes to the outer surface of the tubing 61 through the irrigation ports 80 (FIG. 5A). The fluid is then dispersed over generally the entire surface of the ablation assembly 17 by the porous covering 84. This irrigation enables creation of deeper lesions.

In use, the catheter 10 is inserted into the patient through a suitable guiding sheath whose distal end is positioned at a desired mapping or ablating location. An example of a suitable guiding sheath for use in connection with the present invention is the Preface™ Braided Guiding Sheath, commercially available from Biosense Webster, Inc. (Diamond Bar, Calif.). The distal end of the sheath is guided into one of the atria. A catheter in accordance with the present invention is fed through the guiding sheath until its distal end extends out of the distal end of the guiding sheath. As the catheter 10 is fed through the guiding sheath, the tip assembly 17, the flexible section 19 and the intermediate section 14 are generally straightened to fit through the sheath. Once the distal end of the catheter is positioned at the desired mapping or ablating location, the guiding sheath is pulled proximally, allowing the deflectable intermediate section 14, the flexible section 19 and the tip assembly 17 to extend outside the sheath, and return to their original preformed shapes with the tip assembly 17 extending from the intermediate section 14 at a predetermined off-axis angle θ and/or off-plane angle γ.

In one embodiment, where the catheter is advanced into the right atrium, the intermediate section 14 is deflected to approximate the generally convex curvature of the cavo-tricuspid isthmus or the His region where the intermediate section 14 can rest on the tissue and is stabilized and in synch with the motion of the heart.

With the intermediate section 14 deflected, the tip assembly 17 makes contact with tissue in the region by means of the preset off-axis and/or off-plane angle(s) provided by the flexible section 19. To create generally focal lesions during ablation, the ablation assembly is positioned and the flexible section 19 allows the ablation assembly to be readily displaced from contact with the tissue before damage can occur from perforation, steam build-up and the like. For continuous lesions during ablation, the tip assembly 17 is dragged along the tissue surface. As the ablation assembly encounters uneven formation such as a projection or recess in the tissue surface, the flexible section 19 flexes as the ablation assembly 17 pivots from the preset angle(s) to absorb the movement without affecting the intermediate section 14. The catheter body may also be rotated to form a linear line of block at the His region. Because the off-plane angle allows the ablation assembly to reach tissue lateral of the plane of deflection, rotation of the ablation assembly (e.g., by rotation of the catheter body and/or the control handle) can create a generally linear ablation line.

Regardless of the ablation lesion desired, the tip assembly 17 maintains continuous contact with the tissue for improved lesions. In the embodiment of the catheter for mapping applications, similar manipulations of the catheter and the control handle enable the mapping electrodes 85A, 85b, 86a, 86b and 86c to map in a linear or circumferential pattern.

The preceding description has been presented with reference to presently preferred embodiments of the invention. Workers skilled in the art and technology to which this invention pertains will appreciate that the Figures are not necessarily to scale and alterations and changes in the described structure may be practiced without meaningfully departing from the principal spirit and scope of this invention. Accordingly, the foregoing description should not be read as pertaining only to the precise structures described and illustrated in the accompanying drawings, but rather should be read consistent with and as support for the following claims which are to have their fullest and fairest scope.

What is claimed is:

1. A catheter comprising:
an elongated flexible tubular catheter body;
an intermediate section attached to a distal end of the catheter body, the intermediate section having a first flexural modulus;
an ablation assembly including an electrode and an electromagnetic sensor, and
a flexible section connecting the ablation assembly to the intermediate section at a preset angle, the flexible section having a second flexural modulus that is less than the first flexural modulus.

2. The catheter of claim 1, wherein the electrode is a coil electrode.

3. The catheter of claim 2, wherein the coil electrode has a length ranging from abut 8 mm to about 15 mm.

4. The catheter of claim 2, further comprising an irrigation tube and a plurality of irrigation ports disposed along the ablation assembly.

5. The catheter of claim 4, wherein the irrigation tube terminates in the ablation assembly, proximal to the plurality of irrigation ports.

6. The catheter of claim 5, further comprising a porous covering disposed over the coil electrode and the irrigation port.

7. The catheter of claim 6, wherein the porous covering comprises expanded polytetrafluoroethylene.

8. The catheter of claim 6, further comprising:
a proximal ring electrode disposed on a proximal region of the ablation assembly about the porous covering; and
a distal ring electrode disposed on a distal region of the ablation assembly about the porous covering.

9. The catheter of claim 8, further comprising a puller wire having a distal end connected to a distal end of the intermediate section, and a proximal end connected to a control handle attached to a proximal end of the catheter body.

10. The catheter of claim 9, wherein the preset angle includes an off-axis angle.

11. The catheter of claim 10, wherein the off-axis angle is between about ten degrees to about sixty degrees.

12. The catheter of claim 10, wherein the preset angle further includes an off-plane angle.

13. The catheter of claim 12, wherein the off-plane angle is between about twenty degrees and about ninety degrees.

14. The catheter of claim 13, wherein the off-axis angle is about twenty degrees and the off-plane angle is about ninety degrees.

15. The catheter of claim 12, wherein the second flexural modulus is between about one-quarter to about one-half of the first flexural modulus.

16. The catheter of claim 12, wherein the intermediate section has a first durometer measurement and the flexible section has a second durometer measurement that is between about one-quarter to about one-half of the first durometer measurement.

17. A method for ablating tissue at or near a generally convex region of a right atrium of a heart, the method comprising:
inserting into the region a distal end of a catheter including,
an elongated flexible tubular catheter body having proximal and distal ends,
at least a portion of an intermediate section attached to the distal end of the catheter body, the intermediate section having a first flexural modulus,
an ablation assembly including an ablation electrode, and
a flexible section connecting the ablation assembly to the intermediate section at a preset angle, the flexible section having a second flexural modulus that is less than the first flexural modulus;
deflecting the intermediate section such that the ablation assembly contacts a surface of the region;
moving the ablation assembly along the surface;
applying energy to the ablation electrode; and
changing the preset angle to another angle without displacing the intermediate section.

18. The method of claim 17, further including creating a continuous lesion on the surface.

19. The method of claim 18, wherein the step of deflecting the intermediate section causes the intermediate section to conform to the surface.

20. The method of claim 19, wherein the step of moving the ablation assembly along the surface includes moving the ablation assembly along the surface in a linear direction.

21. The method of claim 20, wherein the ablation assembly further comprises a mapping electrode.

22. The method of claim 21, further comprising recording electrograms from the ablation assembly.

23. The method of claim 22, wherein the ablation assembly further includes a plurality of irrigation ports and a porous covering disposed over the plurality of irrigation ports.

24. The method of claim 23, further comprising dispersing an irrigation fluid through the porous covering.
